(19) United States
(12) Patent Application Publication
(10) Pub. No.: US 2015/0174145 A1
(43) Pub. Date: Jun. 25, 2015

(54) Pharmaceutical compounds with the structure of ionic salts with antibiotic and anticancer action

(71) Applicant: Pedro Brito Correia, Estoril (PT)

(72) Inventor: Pedro Brito Correia, Estoril (PT)

(21) Appl. No.: 14/138,156

(22) Filed: Dec. 23, 2013

(51) Int. Cl. A61K 31/66; A61K 31/14; A61K 31/4425; A61K 31/4164; A61K 31/43; A61K 31/7048

(52) U.S. Cl. CPC A61K 31/66; A61K 31/43; A61K 31/7048; A61K 31/4425; A61K 31/4164; A61K 31/14

(57) ABSTRACT

Pharmaceutical compounds were prepared as ionic salts, liquid under 150° C., consisting in a cation containing Alkyl and/or Phenyl groups and an anion consisting in an existing pharmaceutical antibiotica, whose biological properties are strongly enhanced by the cation, concerning elimination of resistance of bacteria and surprisingly also anticancer action.

### 1. FIELD OF INVENTION

Bacteria resistance to antibiotica, Cancer treatment, Ionic liquids, Pharmaceuticals

### 2. BACKGROUND OF THE INVENTION

In 1971 Monsanto applied for a US patent for a new crop protection product named glyphosate, chemically N-phosphono methyl glycine, and claimed a formulation containing the glyphosate salt of isopropyl amine. This was the first world wide application of an ionic liquid to biological applications, and became in a few years the number one crop protection product sold in the world. It was the herbicide round up. After 1987 this herbicide started to be produced in the European Union.

The isopropylamine salt of glyphosate anion was chosen due to the synergistic action of the cation and anion, concerning the membrane transport, and enzymatic interaction.

The same biological interest exists for pharmaceuticals, when known active substances are converted to an anion which is coupled to a cation containing alkyl groups. This cation influences:
The solubility in the water contained in the biological organism
The solubility in the blood
The transportation velocity through membranes
The interaction with enzymes, reducing the metabolism of the pharmaceutical product
The interaction with enzymes which increase the biological efficiency

In the case of pharmaceutical products used as antibiotica there are today important aspects concerning:
the appearance of resistance in bacteria to antibiotica
the difficulty to find new molecules with antibiotic behaviour and acceptable side effects
the possibility to use antibiotica as inhibitors of cancer cells growth

### 3. DETAILED DESCRIPTION OF THE INVENTION

We compared the biological effects for following antibiotica:
A—Penicillin-3 (2S,5R,6R)-3,3-dimethyl-7-oxo-6-(2-phenylacetmido)-4-thia-1-azabicyclo(3.2.0)heptane-2-carboxylate. We show below the ammonium salt

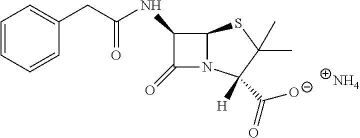 [SMILES: CC1(C)S[C@@H]2[C@H](NC(=O)Cc3ccccc3)C(=O)N2[C@H]1C(=O)[O-] ; [NH4+]]

B—Ampicillin-(2S,5R,6R)-6-(®-2-amino-2-phenylacetamido)-3,3-dimethyl-7-oxo-4-thia-1-azabicyclo(3.2.0)heptane-2-carboxylate. Below we show the phosphonium salt:

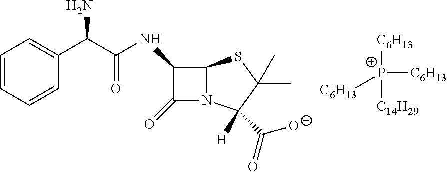

C—Amoxicillin-1-ethyl-3-methyl-1H-imidazol-3-ium (2S,5R,6R)-6-R)-2-amino-2-(4-hydroxyphenyl) acetamido)-3,3-dimethyl-7-oxo-4-thia-1-azabicyclo(3.2.0)heptane-2-carboxylate Below we show the imidazolium salt:

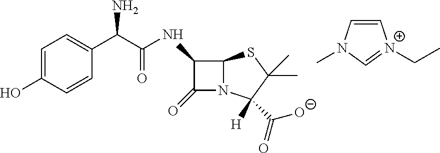

D—Amphotericin (1R,3S,5R,6R,9R,11R,15S,16R,17R,18S,19E,23E,25E,27E,29E,31E,33R,35S,36R,37S)-33-(((2R,3S,4S,5S,6R)-4-amino-3,5-dihydroxy-6-methyltetrahydro-2H-pyran-2-yl)oxy)-1,3,5,6,9,11,17,37-octahydroxy-15,16,18-trimethyl-13-oxo-14,39-dioxabicyclo(33.3.1)nonatriaconta-19,21,23,25,27,29,31-heptaene-36-carboxylate We show below the pyridinium salt

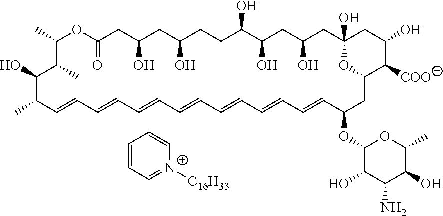

Each one of these 4 antibiotica were converted to anions and coupled to each one of the 6 following cations:
1—1-ethyl-3-methylimidazolium
2—Hydroxyl-ethyl-3-methylimidazolium
3—Choline
4—Tetraethyl ammonium
5—Cetylpyridinium
6—Trihexyltetradecylphosphonium

The combinations of the 4 antibiotica with the 6 cations produced 24 ionic liquids whose biological behaviour was tested in comparison with the corresponding commercial forms of the antibiotica, which are acid, sodium salt or ammonium salt.

The ionic liquids could be produced using the method as described in reference 2, or starting from the sodium salt and using amberlite ion exchange columns as described in reference 1.

The patent in reference 2 is a process and a product patent describing a well known general procedure to obtain ionic liquids by mixing the compounds containing the cation and anion of the ionic liquid and purifying. No results are presented concerning biological efficacy. There are 30 claims concerning process or production methods and 163 product claims, including almost all pharmaceuticals, agrochemicals and nutritional substances, without making any biological tests.

A patent claiming for a pharmaceutical use, the application of every chemical compound, would not be accepted, without a justification of the biological efficacy of each compound. Otherwise it would not be interesting for any producer to spend money making biological research for a product which can be no more protected by a patent.

Our patent application is a product patent describing tests on biological efficacy and claiming their medical use, independently of the production process.

Each one of the 24 combinations of the ionic liquids were tested with following 9 bacteria:
Escherichia coli ATCC25922
Staphylococcus aureus ATCC25923
Klebsiella pneumonia
Staphylococcus epidermidis
Enterococcus fecalis
E. coli Tem CTXM9
E. coli CTXM2
E. coli AmpCMox2
Saccharomyces cerevisiae PYCC 4072

These bacteria were tested in Vitek2 systems from bioMérieux. Bacteria were grown on Trypticase soy agar for 24 hours at 37° C. prior to each test. The bacteria assays were made in Tryptic Soy Broth.

All ionic liquids from antibiotic were prepared according to the process described in literature (1).

All 24 ionic liquids were tested also in 7 human cancer cells, namely:
T47D—ductal breast epithelial tumour
PC3—Prostate cancer
HepG2—hepatocellular liver carcinoma
MG63—osteosarcoma
RKO—colon cancer
SF—skin fibroblasts
GF—gingival fibroblasts

### Example 1

### Synthesis of tetraethyl ammonium salt of Ampicillin

Tetraethylammonium bromide (0.321 r, 1.53 mmol) was dissolved in methanol and passed through an ion exchange column Amberlite IRA-400-OH (5 eq. flux rate 0.133 mlml⁻¹ min⁻¹=8 BVh⁻¹). Then, the tetraethylammonium hydroxide solution, coming out from the ion exchange column, was slowly added to ampicillin (0.549 g 1.57 mmol) dissolved in 1.0 M ammonium solution (50 mg ml⁻¹). The reaction mixture was stirred at room temperature for 1 hour. After solvent evaporation, the residue was dissolved in 20 ml of methanol acetonitrile 1:9 and left refrigerated overnight at 4° C. to induce crystallization of excess of the ampicillin. Then the ampicillin salt crystals were filtered from the solution and dried in vacuum for 24 hour. The product has a melting point of 79° C.

Example 2 Cells were maintained in alpha minimal essential medium containing 10% fetal bovine serum, 100 Iuml⁻¹ penicillin, 2.5 microg ml⁻¹ streptomycin, 2.5 microg ml⁻¹ amphotericin B and 50 microg ml⁻¹ ascorbic acid. At about 70-80% confluence, cells were enzymatically detached with 0.05% trypsin and 0.5 mM EDTA and seeded at 10⁴ cells cm⁻².

After an attachment period of 24 hours, the culture medium was renewed, and supplemented with different concentrations (0.005-500 mM) of the ampicillin derived ionic liquid. Cell cultures were maintained in a 5% CO₂ humidified atmosphere at 37° C.

Cellular viability/proliferation was assessed by the MTT assay at days 1, 3 and 5 of the culture. This assay is based on the reduction of 3-(4,5-dimethylthiazol-2-yl)-2,5-diphenyltetrazolium bromide (MTT) to a purple formazan product by viable cells.

Shortly, cultured cells were incubated at 37° C. with 0.5 mg ml −1 of MTT for 4 hours. The culture medium was then removed; the stained product dissolved with dimethylsulfoxide, and absorbance determined at 550 nm in an ELISA plate rewader. Results were expressed as absorbance per cm2.

The values of the maximal inhibitory concentration (IC50) and the median lethal dose (LD50) were obtained from nonlinear regression analysis of concentration effect curves using the GGraphPad Prism Software.

The definition of IC50 is the response corresponding to the 50% control, considered as the mean of the 0% and 100% assay controls. It is used to measure the efficacy of a compound in inhibiting any biochemical or biological function.

The term LD50 means the median lethal dose and is the amount of material which causes the death of 50% of a population.

The results obtained are indicated in tables 1 to 7:

TABLE 1
Minimum inhibitory concentrations (mM) on the
ampicillin sensitive bacterial strains tested.

| | Strains | | | | |
|---|---|---|---|---|---|
| | Gram-negative | | Gram-positive | | |
| | E. coli | | S. aureus | | |
| Comp. | ATCC 25922 | K. pneumoniae | ATCC 25923 | E. fecalis | S. epidermis |
| [Na][Amp]^{a)} | 0.05 | 2.5 | 0.005 | 0.05 | 0.05 |
| [TEA][Amp] | >5 | >5 | >5 | >5 | >5 |
| [TEA][Br] | >5 | >5 | 2.5 | >5 | >5 |
| [P_{6,6,6,14}][Amp] | 2.5 | 5 | 0.05 | 0.05 | 0.05 |
| [P_{6,6,6,14}][Cl] | 2.5 | 2.5 | 2.5 | >5 | 2.5 |
| [C₁₆Pyr][Amp] | 0.5 | 0.05 | 0.005 | 0.005 | 0.005 |
| [C16Pyr][Cl] | 0.5 | 2.5 | 0.5 | 0.5 | 2.5 |
| [Cholin][Amp] | >5 | >5 | >5 | >5 | >5 |
| [Cholin][Cl] | >5 | >5 | 2.5 | >5 | >5 |
| [EMIM][Amp] | >5 | >5 | >5 | >5 | >5 |
| [EMIM][Br] | >5 | >5 | 0.05 | >5 | 5 |
| [C₂OHMIM][Amp] | 5 | >5 | >5 | 5 | 2.5 |
| [C₂OHMIM][Cl] | 5 | >5 | >5 | 5 | 5 |

^{a)}The [Na][Amp] was used as control.

TABLE 2
Minimum inhibitory concentrations (mM) on
the ampicillin resistant bacterial strains tested.

| | Strains | | |
|---|---|---|---|
| | E. coli | E. coli | E. coli |
| Comp. | TEM CTX M9 | CTX M2 | AmpC MOX2 |
| [Na][Amp]^{a)} | >5 | >5 | >5 |
| [TEA][Amp] | >5 | >5 | >5 |
| [TEA][Br] | >5 | 2.5 | >5 |
| [P_{6,6,6,14}][Amp] | 0.5 | 0.5 | >5 |
| [P_{6,6,6,14}][Cl] | 2.5 | 5 | >5 |
| [C₁₆Pyr][Amp] | 0.005 | 0.05 | >5 |
| [C16Pyr][Cl] | 0.5 | >5 | >5 |
| [Cholin][Amp] | >5 | >5 | >5 |
| [Cholin][Cl] | >5 | >5 | >5 |
| [EMIM][Amp] | >5 | >5 | >5 |
| [EMIM][Br] | 5 | >5 | >5 |
| [C₂OHMIM][Amp] | >5 | >5 | 5 |
| [C₂OHMIM][Cl] | >5 | 2.5 | 5 |

^{a)}The [Na][Amp] was used as control.

TABLE 3
Relative Decrease of Inhibitory Concentration (RDIC) of ampicillin anion in [Cat][Amp]
(RDIC) comparing with sodium ampicillin for ampicillin sensitive bacteria^{a)}.

| | Strains | | | | |
|---|---|---|---|---|---|
| | Gram-negative | | Gram-positive | | |
| | E. coli | | S. aureus | | |
| Comp. | ATCC 25922 | K. pneumoniae | ATCC 25923 | E. fecalis | S. epidermis |
| [Na][Amp]^{a)} | 1 | 1 | 1 | 1 | 1 |
| [P_{6.6.6.14}][Amp] | 0.02 | 0.5 | 0.1 | 1 | 1 |
| [C₁₆Pyr][Amp] | 0.1 | 50 | 1 | 10 | 10 |
| [C₂OHMIM][Amp] | 0.01 | — | — | 0.01 | 0.02 |

^{a)}for explanation of RDIC values see Experimental section.

TABLE 4
Relative Decrease of Inhibitory Concentration (RDIC)^{a)} of ampicillin
anion in [Cat][AMP] comparing with Sodium Ampicillin for
ampicillin resistant bacteria^{a).}

| | Strains | | |
|---|---|---|---|
| | E. coli | | |
| Comp. | TEM CTX M9 | E. coli CTX M2 | E. coli AmpC Mox2 |
| [Na][Amp] | 1 | 1 | 1 |
| [P_{6,6,6,14}][Amp] | >10 | >10 | — |
| [C₁₆Pyr][Amp] | >1000 | >100 | — |

^{a)}for explanation of RDIC values see Experimental section.

TABLE 5
Growing rates for the different organisms in the presence and absence of [P_{6,6,6,14}][Amp].

| | MIC/ | Conc. Tested/ | Growing rate · min⁻¹ | | |
|---|---|---|---|---|---|
| Organism | mM | mM | With [P_{6,6,6,14}][Amp] | no compound | Decreasing rate |
| E. coli ATCC 25922 | 2.5 | 0.5 | 0.0007 ± 0.0001 | 0.0020 ± 0.0001 | 0.035 |
| S. aureus ATCC 25923 | 0.005 | 0.0005 | 0.0004 ± 0.0003 | 0.0052 ± 0.0007 | 0.077 |
| K. pneumonia | 5.0 | 0.5 | 0.00045 ± 0.00007 | 0.0025 ± 0.0005 | 0.18 |
| E. fecalis | 0.05 | 0.005 | 0.0009 ± 0.0004 | 0.0033 ± 0.0001 | 0.28 |
| S. epidermis | 0.05 | 0.005 | 0.0006 ± 0.0000 | 0.0047 ± 0.0002 | 0.13 |
| E. coli TEM CTX M9 | 0.5 | 0.05 | 0.00067 ± 0.00006 | 0.0033 ± 0.0007 | 0.20 |
| E. coli AmpC Mox2 | 0.5 | 0.05 | 0.00077 ± 0.00006 | 0.0032 ± 0.0004 | 0.24 |

TABLE
Erro! Não existe nenhum texto com o estilo
especificado no documento. IC50 and LD50 in μMof the
ILs based on ampicillin against primary human cell lines.

| | Cell lines | |
|---|---|---|
| Compounds | SF | GF |

| | | | |
|---|---|---|---|
| [TEA][Amp] | IC50/μM | nd | nd |
| | LD50/μM | nd | 58.040 |
| [P_{6,6,6,14}][Amp] | IC50/μM | 0.249 | 0.173 |
| | LD50/μM | >0.249 | 0.176 |
| [C₁₆Pyr][Amp] | IC50/μM | 0.032 | 0.012 |
| | LD50/μM | 0.815 | 45.510 |
| [Cholin][Amp] | IC50/μM | 48.480 | nd |
| | LD50/μM | 49.790 | nd |
| [EMIM][Amp] | IC50/μM | 6.366 | 0.853 |
| | LD50/μM | >6.366 | 9.357 |
| [C₂OHMIM][Amp] | IC50/μM | 5.084 | 0.462 |
| | LD50/μM | 22.600 | 30.470 |
| [Na][Amp]* | IC50/μM | nd | 109.100 |
| | LD50/μM | 8.104 | >109.100 |

nd - IC50 and LD 50 not detected in the concentration range used.
SF (skin fibroblasts), gingival fibroblasts (GF).
[Amp] Ampicillin anion, [TEA] tetraethylammonium, [P_{6,6,6,14}] trihexyltetradecylphosphonium, [C₁₆pyr] cetylpyridinium, [Cholin] choline, [EMIM] 1-ethyl-3-methylimidazolium, [C₂OHMIM] 1-hydroxy-ethyl-3-methylimidazolium.
*[Na][Amp] was used as a control.

TABLE 7
IC50 and LD50 in μM of the ILs based on ampicillin against cancer cell lines.

| | | Cell lines | | | | |
|---|---|---|---|---|---|---|
| Compounds | | MG63 | HepG2 | T47D | PC3 | RKO |
| [TEA][Amp] | IC50/μM | 0.030 | nd | 0.042 | 35.650 | 56.700 |
| | LD50/μM | 1.368 | 0.0951 | 43.990 | 58.920 | 68.370 |
| [P_{6,6,6,14}][Amp] | IC50/μM | 0.312 | 0.322 | 0.264 | 0.354 | 0.180 |
| | LD50/μM | >0.312 | 15.770 | 4.970 | 9.335 | 11.860 |
| [C₁₆Pyr][Amp] | IC50/μM | 0.011 | nd | 0.005 | 132.700 | 0.226 |
| | LD50/μM | 183.200 | nd | nd | >132.700 | 59.170 |
| [Cholin][Amp] | IC50/μM | 0.017 | 1.619 | 110.900 | 0.982 | nd |
| | LD50/μM | 738.000 | 15.030 | >110.900 | 17.160 | 0.707 |
| [EMIM][Amp] | IC50/μM | 1.122 | 24.270 | nd | 29.990 | 0.269 |
| | LD50/μM | >1.122 | >24.270 | 32.370 | 31.020 | >209.700 |
| [C₂OHMIM][Amp] | IC50/μM | 0.738 | 0.319 | 0.146 | 0.297 | 0.359 |
| | LD50/μM | 4.197 | 0.4040 | 0.498 | >0.297 | 0.362 |
| [Na][Amp]* | IC50/μM | nd | nd | nd | 0.597 | 2.406 |
| | LD50/μM | 506.600 | 0.720 | 47.790 | 560.900 | 920.800 |

nd - IC50 and LD 50 not detected in the concentration range used.
MG63 (osteosarcoma cell line), HepG2 (hepatocellular liver carcinoma cell), T47D (ductal breast epithelial tumour cell line), PC3 (prostate cancer cell line), line) and RKO (colon cancer cell line).
[Amp] Ampicillin anion, [TEA] tetraethylammonium, [P_{6,6,6,14}] trihexyltetradecylphosphonium, [C₁₆pyr] cetylpyridinium, [Cholin] choline, [EMIM] 1-ethyl-3-methylimidazolium, [C₂OHMIM] 1-hydroxy-ethyl-3-methylimidazolium.
*[Na][Amp] was used as a control.

We conclude from these tables and similar tables prepared for the other compounds referred before that:
the antibiotic effect on bacteria was specially enhanced when using the cations hydroxyl-ethyl-3-methylimidazolium or alternatively as a cation cetyl pyridiniumn or trihexyltetradecylphosphonium thereby providing a way to be effective even in case of strains of bacteria resistant to classical antibiotics.
The antitumoral effect was specially strong in case of the composition having as the anion the ampicillin and as a cation hydroxyl-ethyl-3-methylimidazolium or alternatively as a cation cetyl pyridiniumn or trihexyltetradecylphosphonium.
By biological similarity, we considered that the same effect of the cations is expected for the anions obtained from following compounds:

Phenoxymethylpenicillin (penicillin V); Metampicillin; Benzylpenicillin (penicillin G), Flucloxacillin; Dicloxacillin; Meticillin; Azlocillin; Carbenicillin; Cloxacillin; Ticarcillin; Dicloxacillin; Mezlocillin; Nafcillin; Oxacillin; Piperacillin, Temocillin.

1. A pharmaceutical product consisting of an ionic liquid with melting temperature under 150° C. with anions based on Penicillin, Ampicillin, Amoxicillin, Amphotericin, Phenoxymethylpenicillin (penicillin V); Metampicillin; Benzylpenicillin (penicillin G), Flucloxacillin; Dicloxacillin; Meticillin; Azlocillin; Carbenicillin; Cloxacillin; Ticarcillin; Dicloxacillin; Mezlocillin; Nafcillin; Oxacillin; Piperacillin, Temocillin.
and cations based on cetyl pyridinium, trihexyltetradecylphosphonium, 1-ethyl-3-methylimidazolium, hydroxyl-ethyl-3-methylimidazolium, choline, tetraethyl ammonium, which increase the antibiotic effect of the compounds corresponding to the anions, eliminating the problems of bacteria resistant to antibiotic, and impairing to the normal antibiotica a surprising effect on cancer cells.

2. The compositions of claim 1, wherein the antitumoral effect was specially strong in case of the composition having as the anion the ampicillin and as a cation hydroxyl-ethyl-3-methylimidazolium or alternatively as a cation cetyl pyridiniumn or trihexyltetradecylphosphonium.

3. The compositions of claim 1, wherein the antibiotic effect on bacteria was specially enhanced when using the cations hydroxyl-ethyl-3-methylimidazolium or alternatively as a cation cetyl pyridiniumn or trihexyltetradecylphosphonium thereby providing a way to be effective even in case of strains of bacteria resistant to classical antibiotics.
